(19) United States
(12) Patent Application Publication
(10) Pub. No.: US 2023/0272332 A1
(43) Pub. Date: Aug. 31, 2023

(54) METHOD AND SYSTEM FOR COLONY PICKING

(71) Applicant: NATIONAL UNIVERSITY OF SINGAPORE, Lower Kent Ridge Road (SG)

(72) Inventors: Ming WANG, (TW); Wai Kit CHEE, Lower Kent Ridge Road (SG); Chueh Loo POH, Lower Kent Ridge Road (SG); Chwee Teck LIM, Lower Kent Ridge Road (SG)

(73) Assignee: NATIONAL UNIVERSITY OF SINGAPORE, Lower Kent Ridge Road (SG)

(21) Appl. No.: 18/012,660

(22) PCT Filed: Jun. 24, 2021
(86) PCT No.: PCT/SG2021/050368

§ 371 Date: Dec. 23, 2022

(30) Foreign Application Priority Data
Jun. 24, 2020 (SG) 10202006074Y

(51) Int. Cl. C12N 1/02; C12N 1/20; C12M 1/34; C12M 1/00; B01L 3/00

(52) U.S. Cl. CPC C12N 1/02; C12N 1/20; C12M 41/36; C12M 47/04; B01L 3/502761; B01L 2200/0673; B01L 2300/0816; B01L 2200/0652; B01L 2300/0654; B01L 2300/0864

(57) ABSTRACT

The present disclosure relates to a method of colony picking. The method includes the steps of mixing a bacterial suspension with an oil-based carrier liquid for generating a plurality of droplets comprising bacteria contained in the bacterial suspension and incubating the plurality of droplets for a predetermined period of time to allow growth of bacteria within the plurality of droplets. The method further includes the steps of screening each of the plurality of droplets that flows through one or more microfluidic channels of a microfluidic device to determine an opacity degree of each of the plurality of droplets, wherein the opacity degree is indicative of colony formation in the plurality of droplets, and sorting the plurality of droplets based on the opacity degree of each of the plurality of droplets.

### FIELD OF INVENTION

The present invention relates broadly, but not exclusively, to a method and system for colony picking.

### BACKGROUND

The process of introducing DNA (and other similar polynucleotides) into host cells is a key aspect of recombinant DNA technology. The process by which polynucleotides are introduced into host cells is called transformation. Bacterial cells generally remain the preferred hosts for the majority of recombinant DNA experiments and genetic engineering manipulations.

Conventional methods of culturing and selecting bacterial cells typically involve culturing transformed bacterial cells on antibiotic-containing agar plates. Only bacterial cells that contain the introduced DNA would be able to divide and grow and form colonies in the presence of the antibiotic. Colony picking and/or colony counting will then be performed before carrying out downstream procedures.

Colony picking and counting procedures are tedious and time consuming. Efforts have been made to automate these procedures mainly through the development of automated colony pickers. By using image analysis techniques and internal algorithms to differentiate actual colonies from background noise caused by bubbles and colony irregularities, the colonies formed on the agar plates can be counted and picked by robotic-controlled sterilized pins and segregated into individual wells (typically of 96 and 384 well plates) for culturing. However, the use of automated colony pickers is limited by the high costs of the equipment, the amount of reagents used (which is similar to the amount used in manual methods), the risk of contamination, and the level of equipment maintenance required. In addition, the low throughput level (on average about 1000 colonies/hour) makes it difficult for the automated colony pickers to keep up with the ever expanding need for large scale genetic constructions.

Thus, there is a need for an automated method of colony picking and/or counting that circumvents the problems associated with the existing colony picking and/or counting procedures.

A need therefore exists to provide a system and method that seek to address at least one of the problems above or to provide a useful alternative.

### SUMMARY

According to a first aspect of the present invention, there is provided a method of colony picking, the method comprising the steps of:
mixing a bacterial suspension with an oil-based carrier liquid for generating a plurality of droplets comprising bacteria contained in the bacterial suspension;
incubating the plurality of droplets for a predetermined period of time to allow growth of bacteria within the plurality of droplets;
screening each of the plurality of droplets that flows through one or more microfluidic channels of microfluidic device to determine an opacity degree of each of the plurality of droplets, wherein the opacity degree is indicative of colony formation in the plurality of droplets; and
sorting the plurality of droplets based on the opacity degree of each of the plurality of droplets.

According to a second aspect of the present invention, there is provided a system for colony picking comprising:
a droplet generator for mixing a bacterial suspension with an oil-based carrier liquid to generate a plurality of droplets comprising bacteria contained in the bacterial suspension;
an incubator for culturing the plurality of droplets for a predetermined period of time to allow growth of bacteria within the plurality of droplets;
a microfluidic device comprising one or more microfluidic channels for receiving the incubated droplets, wherein each of the plurality of droplets flows through the one or more microfluidic channels consecutively;
an optical device for screening each of the plurality of droplets that flows through the one or more microfluidic channels to determine an opacity degree of each of the plurality of droplets, wherein the opacity degree is indicative of colony formation in the plurality of droplets; and
a sorting module configured to sort the plurality of droplets based on the opacity degree of each of the plurality of droplets.

### BRIEF DESCRIPTION OF THE DRAWINGS

Embodiments of the invention are provided by way of example only, and will be better understood and readily apparent to one of ordinary skill in the art from the following written description and the drawings, in which:

FIG. 1A shows a diagram illustrating the process of preparing droplets containing bacteria in accordance with an example embodiment.

FIG. 1B shows a diagram illustrating the process of screening the droplets of FIG. 1A for a presence of colony.

FIG. 10 shows a graph illustrating an example of a generated signal in the screening process of the droplets of FIG. 1B.

FIG. 2 shows 2 sets of graphs including screening results associated with droplets with diameters of 40 μm and 100 μm respectively in accordance with an example embodiment.

FIG. 3 shows a flow chart illustrating a method for colony picking in accordance with an example embodiment.

### DETAILED DESCRIPTION

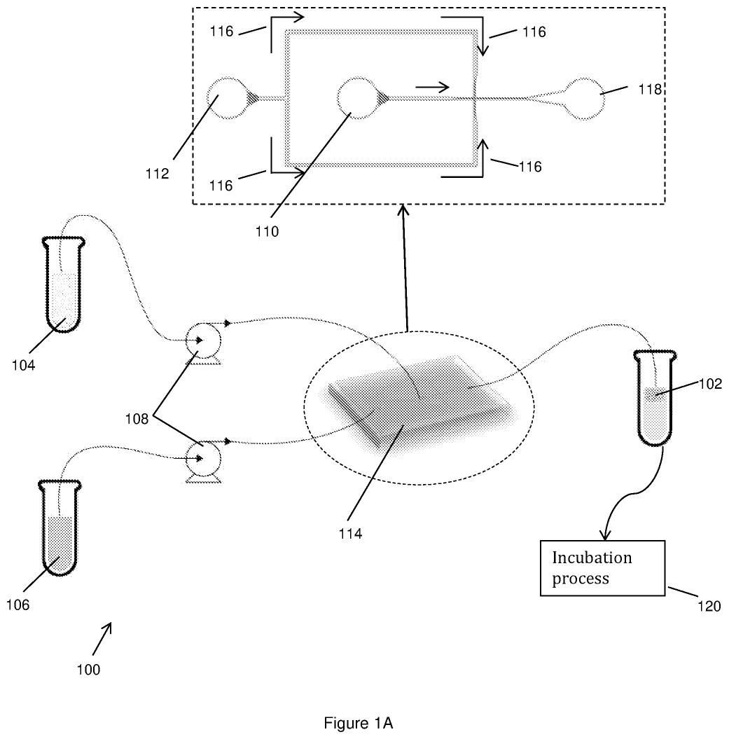

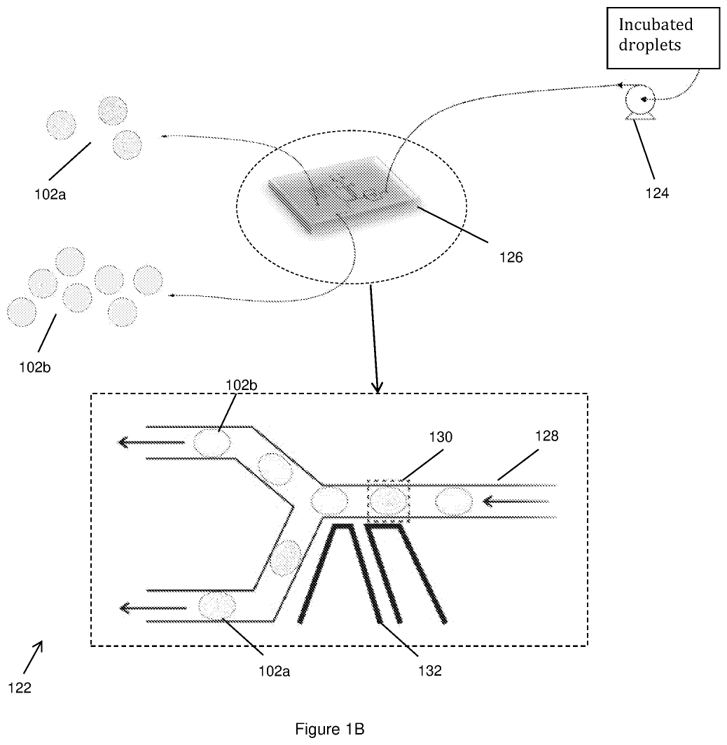

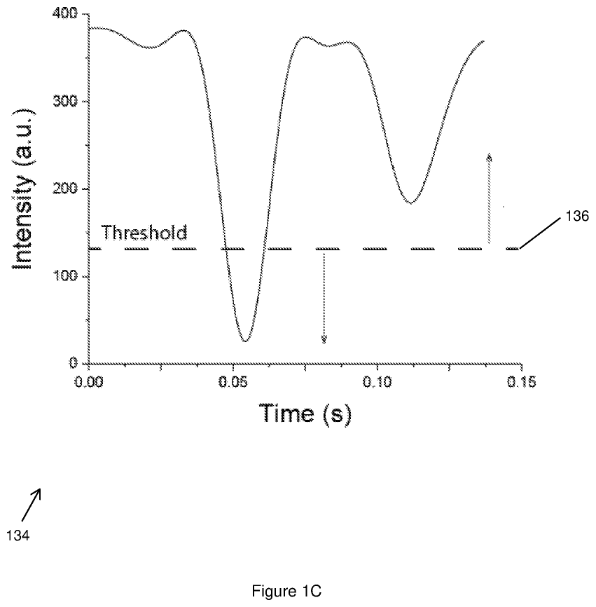

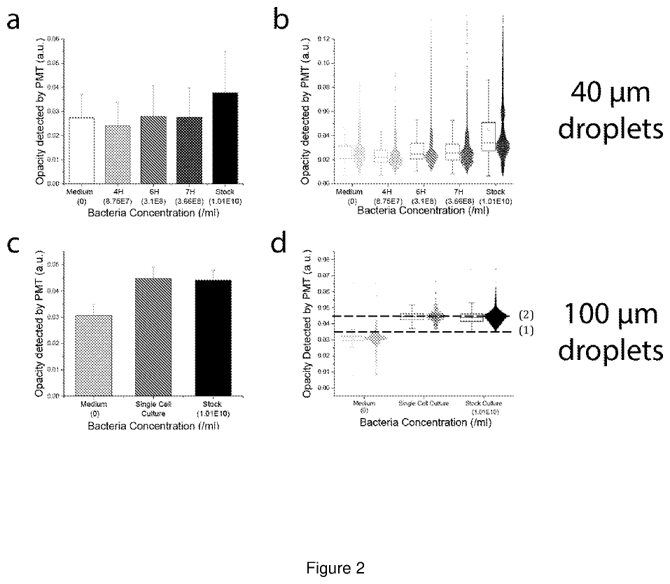

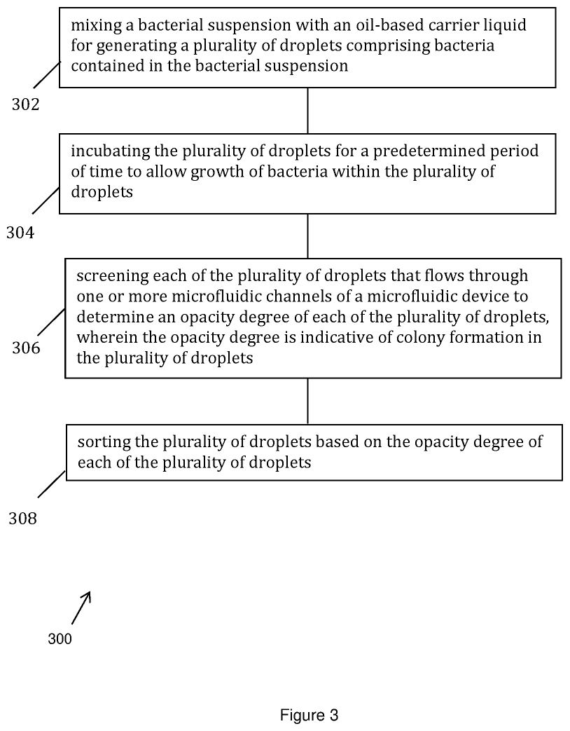

FIG. 1A shows a diagram 100 illustrating the process of preparing droplets 102 containing bacteria in accordance with an example embodiment. A bacterial concentration including a culture medium is prepared and incubated for a predetermined period. The bacterial concentration is then diluted with a culture medium that contains a component suitable to exert a selection pressure on genetically modified bacteria (e.g. antibiotic) to form a bacterial suspension at a predetermined concentration.

Next, each bacterium in the bacterial suspension is encapsulated in single-cell droplets and incubated for a predetermined period before being screened by a microfluidic system for droplets that include microbial colonies. Details of the system and method for colony picking in accordance with an example embodiment are explained in further detail below.

A bacterial suspension 104 is prepared in a first container by diluting a bacterial concentration with a culture medium to a predetermined concentration (e.g. 100 bacteria/ml). The culture medium contains an antibiotic to exert a selection pressure on the bacteria. For example, the initial bacterial concentration contains Escherichia coli DH10β (New England Biolabs) cells that has undergone transformation and grown in Lysogeny broth (LB) medium without antibiotics in the recovery phase. The bacterial concentration is then diluted with the LB medium with antibiotic kanamycin (50 ug/ul) (Sigma) into a bacterial suspension at a predetermined concentration.

The radius of the droplets and the concentration of the bacterial suspension are selected to achieve 1 bacterium per droplet for single-cell encapsulation. Specifically, to achieve an average of 1 bacterium per droplet, the concentration of the bacterial suspension is determined based on the desired radius of the droplets according to the formula

$$\frac{3 \times 1 {0}^{11}}{4 π {r}^{3}} bacteria / ml ,$$

where r is the radius of the droplets in μm.

An oil-based carrier liquid 106 (e.g. NOVEC 7500 fluorocarbon oil (3M, USA) with 0.15% (v/v) oil-based biocompatible surfactant Pico-Surf 1 (Sphere Fluidics, UK)) is prepared in a second container. Pumps 108 (e.g. pressure pump and syringe pump) are used to administer the bacterial suspension 104 and oil-based carrier liquid 106 into a bacterial suspension inlet 110 and an oil-based carrier liquid inlet 112 of a microfluidic droplet generator 114 respectively.

As shown in the enlarged view of the droplet generator 114 in FIG. 1A, the bacterial suspension 104 and the oil-based carrier liquid 106 flows in a direction (shown with arrows 116 in FIG. 1A) towards to a junction, and are mixed to form a plurality of droplets 102 with the bacterial suspension 104 being encapsulated by the oil-based carrier liquid 106. Due to the selection of the radius of the droplets and concentration of the bacterial suspension 104, the generated droplets 102 are mostly empty or contain single bacterium. The generated droplets 102 then flow to an outlet 118 in the droplet generator 114 and collected in a third container.

Next, the collected droplets 102 are placed in an incubator for a predetermined temperature and a predetermined period of time (e.g. 22° C. for 24 hours). Due to the presence of antibiotic in the culture medium of the bacterial suspension 104, only the successfully transformed bacteria which have gene that is resistant to the antibiotic would proliferate and form a monocolony in the droplets 102 during the incubation process 120.

FIG. 1B shows a diagram 122 illustrating the process of screening the droplets 102 of FIG. 1A for a presence of colony. Upon completion of the incubation process, a pump 124 is used to administer the incubated droplets 102 to a microfluidic device 126 for screening and sorting of individual droplets 102. As shown in the enlarged view of the microfluidic device 126 in FIG. 1B, the microfluidic device 126 includes one or more microfluidic channels 128 that allow each of the plurality of droplets 102 to flow through consecutively for the screening and sorting processes to take place. It will be appreciated that the droplets 102 may be collected and incubated in a reservoir (e.g. 1 cm³) that is integrated with the droplet generator 114 and the microfluidic device 126. The reservoir can include a valve operable to allow the droplets 102 to be drained out of the reservoir and flow into the microfluidic channel 128 without using the pump 124.

When the droplets 102 pass through the microfluidic channels 128, an optical device having an objective with a field of view 130 is used to screen each droplet 102 by measuring a bright field intensity associated with the droplets 102 to determine an opacity degree of the droplets 102. The opacity degree of the droplets 102 is indicative of colony formation in the plurality of droplets. The optical device generates a signal based on the measured bright field intensity and transmits the signal to a data acquisition unit for comparison with a predetermined threshold by a sorting module 132.

For example, the microfluidic device 126 is used for screening Escherichia coli cell that harbor an antibiotic resistance plasmid. For cells that have successfully harbored the transformed plasmid, they would grow in the antibiotic-laden medium and expand into a bacterial colony inside the droplets 102, thereby increasing the opacity of the droplets 102 and causing low bright field signal to be generated by the optical device. Opaque droplets 102a which contain the cells could be subsequently sorted away from empty droplets 102b that have low opacity.

The optical device may have two or three components: (1) a light source, (2) a detecting unit, and (3) an amplifying unit to multiply signal from the detecting unit if the signal is weak. In an example, the light source is a halogen light (Leica, Germany), the detecting unit is an inverted microscope DMi8 (Leica, Germany), and the amplifying unit is a photomultiplier tube (PMT) (Hamamatsu, Japan).

Droplets 102 containing bacteria are administered into a long microfluidic channel with a predetermined dimension (e.g. 80×80×90 μm³) at a predetermined flow rate (e.g. 4 μl/min) with a pump (e.g. a syringe pump, Harvard Apparatus, USA). An observation window (e.g. an area of 211.2×211.2 μm²) was continuously shined with halogen light for detecting the bright field intensity when the droplets 102 passed through the microfluidic channel 128. The signal generated based on the bright field intensity is amplified with PMTs and logged into MATLAB for data processing.

The sorting module 132 compares the generated signal from the optical device with a predetermined threshold and transfers the droplets 102 based on the opacity degree of each droplet 102. Specifically, if the intensity signal is lower than the predetermined threshold which suggests that an opaque droplet 102a with successfully transformed bacteria passes through, the sorting module 132 is activated to pull the opaque droplets 102a to a collection outlet. On the other hand, if the intensity signal is higher than the predetermined threshold, the sorting module 132 would allow the empty droplets 102b to passively flow towards a waste outlet.

In an example, the sorting module 132 includes electrodes to synthesize a non-uniform electrical field to pull the opaque droplets by dielectrophoresis. Other examples to actively sort the opaque droplets 102a include (1) operating a membrane valve to pump air/fluid to block a particular channel, (2) generating surface acoustic wave (SAW) to push the opaque droplets 102a and (3) operating a dynamic fluid switch to inject pulse of fluids from side channels to push the opaque droplets 102a. Alternatively, the opaque droplets 102a can be passively sorted (1) based on the content and mass of droplets 102 using gravity or (2) based on difference in size with deterministic lateral displacement (DLD).

FIG. 1C shows a graph 134 illustrating an example of a generated signal in the screening process of the droplets 102 of FIG. 1B. Intensity (a.u.) is plotted on the y-axis and time (s) is plotted on the x-axis. In other words, the graph 134 shows the changes in the intensity signal captured by the objective of the optical device over time in the field of view 130 at the microfluidic channel 128. As shown in the graph, a threshold (represented as reference numeral 136 in FIG. 1C) is predetermined between 100 a.u. and 150 a.u. on the y-axis. The sorting module is configured to compare the intensity signal with the predetermined threshold 136 and transfer the droplets 102 to different groups based on the comparison of the intensity signal with the predetermined threshold 136. In the example shown in FIG. 1C, the intensity signal drops around time 0.05 s, suggesting that an opaque droplet 102a with successfully transformed bacteria is passing through. In that case, the sorting module is activated to pull the opaque droplets 102a to a collection outlet.

Bacterial colony picking/counting remains an essential but mundane task for microbiologists. Efforts have been made to automate the process of colony picking/counting on the plate through the development of automated colony pickers. Nevertheless, automated colony pickers are still limited by the high cost, the risk of contamination, heavy maintenance requirement, and its low throughput.

The system and method for colony picking of the example embodiments involve using integrative microfluidic modules for automation in screening colony formation in droplets 102 that contain bacteria and antibiotic after an incubation process based on the opacity of the droplets. The system and method may also be used to screen enzymes-mutant library for colonies containing unique protein sequences generated by site-directed mutagenesis. Advantageously, the method of the example embodiments can be used for screening for bacteria without any reporter or label since the bacterial colonies are picked based on the opacity of the droplets 102.

The system and method of the example embodiments can facilitate significantly greater throughput (1000 Hz or >million droplets per day) by allowing automated colony picking process while significantly reducing cost (e.g. cost in bio-reagents), time and variability. The bridging of the gap between post-transformation and colony screening processes allows complete chemical isolation between the processes. This may minimise contamination among different colonies and reduce the space required for the colony picking process, as compared to the conventional method which uses agar plates.

FIG. 2 shows 2 sets of graphs (a, b, c and d) including screening results associated with droplets with diameters of 40 μm and 100 μm respectively in accordance with an example embodiment. Depending on the bacteria strains which would result in different proliferation rate, cell size, cell transparency, etc., the degree of change in the opacity of the droplets during an incubation process may vary from one bacterium to another. Thus, droplet size would need to be optimized to yield a more accurate selection.

In the graphs a, b, c and d, opacity detected by PMT (a.u.) is plotted on the y-axis and bacteria concentration (/ml) is plotted on the x-axis for an experiment that involves Escherichia coli. As shown in graphs a and b which illustrate the screening results of 40 μm droplets, opacities (mean opacity in graph a and distribution of opacity in graph b) are compared among droplets with cell culture medium (no cell, negative control), single cell cultured for 4 hours (8.75E7 cell/ml), 6 hours (3.1E8 cell/ml), 7 hours (3.66E8 cell/ml), and stock cell suspension (1.01E10 cell/ml, positive control). There is substantial overlapping in the opacity signals of the droplets of all the different concentrations including droplet that contain only medium and droplets that contain cells in high concentration.

As shown in graphs c and d which illustrate the screening results of 100 μm droplets, opacities (mean opacity in graph c and distribution of opacity in graph d) are compared among droplets with cell culture medium (negative control), single cell culture for 24 hours, and stock cell suspension (1.01E10 cell/ml, positive control). As shown in graph d, there is negligible overlap in the opacity signals of the droplet that contain medium and droplets that contain cells. The wider the range of the overlapping in the opacity signal such as that shown in graphs a and b for 40 μm droplets would cause difficulty in setting the threshold for sorting the droplets. Thus, for Escherichia coli, a larger droplet may facilitate the screening process as large droplets allow more cells to grow and thus a more precise range in opacity which can help in more accurately differentiating droplets containing different concentrations.

A suitable threshold can be set for sorting the droplets with successfully transformed cells from others depending on the applications of the experiments. For example, in graph d, line 1 represents a lower threshold to capture all the positive droplets. Although this allows a higher number of positive droplets to be collected, the collected droplets may contain more false positive (i.e. empty droplets). Line 2 represents a higher threshold to capture droplets with as little negative droplets as possible. Although this reduces the number of negative droplets to be collected, it may compromise the number of positive droplets collected.

FIG. 3 shows a flow chart 300 illustrating a method for colony picking in accordance with an example embodiment. At step 302, a bacterial suspension is mixed with an oil-based carrier liquid for generating a plurality of droplets comprising bacteria contained in the bacterial suspension. At step 304, the plurality of droplets is incubated for a predetermined period of time to allow growth of bacteria within the plurality of droplets. At step 306, each of the plurality of droplets that flows through one or more microfluidic channels of a microfluidic device is screened to determine an opacity degree of each of the plurality of droplets, wherein the opacity degree is indicative of colony formation in the plurality of droplets At step 308, the plurality of droplets is sorted based on the opacity degree of each of the plurality of droplets.

1. A method of colony picking, the method comprising the steps of:
mixing a bacterial suspension with an oil-based carrier liquid for generating a plurality of droplets comprising bacteria contained in the bacterial suspension;
incubating the plurality of droplets for a predetermined period of time to allow growth of bacteria within the plurality of droplets;
screening each of the plurality of droplets that flows through one or more microfluidic channels of a microfluidic device to determine an opacity degree of each of the plurality of droplets, wherein the opacity degree is indicative of colony formation in the plurality of droplets; and
sorting the plurality of droplets based on the opacity degree of each of the plurality of droplets.

2. The method as claimed in claim 1, wherein screening each of the plurality of droplets comprises the steps of:
measuring a bright field intensity associated with each of the plurality of droplets; and
generating a signal based on the measured bright field intensity.

3. The method as claimed in claim 2, wherein screening each of the plurality of droplets comprises detection of light using a photomultiplier (PMT).

4. The method as claimed in claim 3, wherein sorting the plurality of droplets comprises the steps of:
comparing the generated signal with a predetermined threshold; and
transferring the plurality of droplets to different groups based on the comparison of the generated signal with the predetermined threshold.

5. The method as claimed in claim 4, wherein sorting the plurality of droplets comprises separation of the plurality of droplets by dielectrophoresis.

6. The method as claimed in claim 5, further comprising the step of injecting the incubated droplets into the one or more microfluidic channels, wherein each of the plurality of droplets flows through the one or more microfluidic channels consecutively.

7. The method as claimed in claim 6, wherein a radius of the droplets and a concentration of the bacterial suspension are selected to achieve 1 bacterium per droplet.

8. The method as claimed in claim 7, wherein the concentration of the bacterial suspension to be used is determined according to the formula
$$\frac{3 \times 1 {0}^{11}}{4 π {r}^{3}} bacteria / ml ,$$
where r is the radius of the droplets in μm to be used in the method.

9. The method as claimed in claim 8, wherein the suspension comprises bacteria, a bacteria culture medium, and a component suitable to exert a selection pressure on genetically modified bacteria.

10. The method as claimed in claim 9, wherein the oil-based carrier liquid is fluorocarbon oil.

11. A system for colony picking comprising:
a droplet generator for mixing a bacterial suspension with an oil-based carrier liquid to generate a plurality of droplets comprising bacteria contained in the bacterial suspension;
an incubator for culturing the plurality of droplets for a predetermined period of time to allow growth of bacteria within the plurality of droplets;
a microfluidic device comprising one or more microfluidic channels for receiving the incubated droplets, wherein each of the plurality of droplets flows through the one or more microfluidic channels consecutively;
an optical device for screening each of the plurality of droplets that flows through the one or more microfluidic channels to determine an opacity degree of each of the plurality of droplets, wherein the opacity degree is indicative of colony formation in the plurality of droplets; and
a sorting module configured to sort the plurality of droplets based on the opacity degree of each of the plurality of droplets.

12. The system as claimed in claim 11, wherein the optical device is configured to:
measure a bright field intensity associated with each of the plurality of droplets; and
generate a signal based on the measured bright field intensity.

13. The system as claimed in claim 12, wherein the optical device comprises a photomultiplier (PMT).

14. The system as claimed in claim 12, wherein the sorting module is configured to:
compare the generated signal with a predetermined threshold; and
transfer the plurality of droplets to different groups based on the comparison of the generated signal with the predetermined threshold.

15. The system as claimed in claim 14, wherein the sorting module comprises a synthesizer of a non-uniform electrical field to separate the plurality of droplets by dielectrophoresis.

16. The system as claimed in claim 15, wherein a radius of the droplets and a concentration of the bacterial suspension are selected to achieve 1 bacterium per droplet.

17. The system as claimed in claim 16, wherein the concentration of the bacterial suspension is determined according to the formula
$$\frac{3 \times 1 {0}^{11}}{4 π {r}^{3}} bacteria / ml ,$$
where r is the radius of the droplets in μm.

18. The system as claimed in claim 17, wherein the suspension comprises bacteria, a bacteria culture medium, and a component suitable to exert a selection pressure on genetically modified bacteria.

19. The system as claimed in claim 18, wherein the oil-based carrier liquid is fluorocarbon oil.
